(19) United States
(12) Patent Application Publication
(10) Pub. No.: US 2024/0261318 A1
(43) Pub. Date: Aug. 8, 2024

(54) ANTI-AGING COSMETIC COMPOSITION

(71) Applicant: LG HOUSEHOLD & HEALTH CARE LTD., Seoul (KR)

(72) Inventors: Seon Ju LEE, Seoul (KR); Mi Na KIM, Seoul (KR); Seung Hyun JUN, Seoul (KR); Seo Gyeong LEE, Seoul (KR)

(73) Assignee: LG HOUSEHOLD & HEALTH CARE LTD., Seoul (KR)

(21) Appl. No.: 18/566,308

(22) PCT Filed: Jun. 2, 2022
(86) PCT No.: PCT/KR2022/007823

§ 371 Date: Dec. 1, 2023

(30) Foreign Application Priority Data
Jun. 4, 2021 (KR) 10-2021-0072613

(51) Int. Cl. A61K 31/7076; A61K 8/49; A61K 8/60; A61K 31/341; A61K 31/352; A61P 17/00; A61Q 19/08

(52) U.S. Cl. CPC A61K 31/7076; A61K 8/498; A61K 8/602; A61K 31/341; A61K 31/352; A61P 17/00; A61Q 19/08

(57) ABSTRACT

Provided is a composition for strengthening the dermal layer of the skin and improving skin elasticity or alleviating skin wrinkles by strengthening the basement membrane of the skin. The composition according to the presently claimed subject matter contains a compound represented by chemical formula 1; and a compound represented by chemical formula 2 and/or a compound represented by chemical formula 3, at the same time, and thus has superior skin elasticity or wrinkle alleviation effect than a composition containing the compound represented by chemical formula 1 alone, the compound represented by chemical formula 2 alone, or the compound represented by chemical formula 3 alone.

### CROSS-REFERENCE TO RELATED APPLICATIONS

This application is a 371 of PCT/KR2022/007823 filed on Jun. 2, 2022, which claims the benefit of priority from Korean Patent Application No. 10-2021-0072613 filed on Jun. 4, 2021, the contents of each of which are incorporated herein by reference in its entirety.

### TECHNICAL FIELD

The present invention relates to a composition which strengthens the dermal layer of skin and improves skin elasticity or skin wrinkles by strengthening the basement membrane of the skin.

### BACKGROUND ART

Collagen is a major structural protein produced in dermal fibroblasts and is present in the extracellular matrix. Collagen is known to play an important role in relation to the mechanical firmness of skin, the resistance and adhesion of connective tissue, the support of cell adhesion, and the induction of cell division and differentiation (during organism growth or wound healing). Collagen is reduced due to natural aging resulting from an increase in age or photoaging resulting from UV irradiation, and the reduction is promoted by the activity of collagenase, which is an enzyme that breaks down collagen. It is known that a reduction in collagen is closely associated with skin elasticity degradation or wrinkle formation. Collagen is a fibrous protein commonly found in mammals and constitutes bones, cartilage, the basement membrane, and the like in the body.

Among those listed above, the basement membrane of the skin plays an important role in supporting and adhering epidermal cells, moving nutrients, regulating epidermal differentiation, and the like. When the skin basement membrane is damaged due to aging, flattening, multiplication, insulation and the like of the basement membrane occur, and thus wrinkles are formed. Also, there is an increased possibility that external risk factors may penetrate into the dermis, and thus the skin may be easily damaged. Therefore, in order to restore the damaged skin basement membrane or maintain the same in a healthy state, first, the components thereof need to be properly maintained.

Meanwhile, fibroblast growth factors (FGFs) are one of the growth factors that regulate the proliferation, migration, differentiation, and survival of various cells, and are composed of a group of 23 proteins. FGFs are produced in keratinocytes, fibroblasts, vascular endothelial cells, smooth muscle cells, chondrocytes, mast cells, and the like, and the above-described functions are performed by activating 4 types of FGF receptors expressed in various cells. Particularly, FGF 2, FGF 7, and FGF 10 are known to play an important role in skin regeneration (Non-Patent Document 1). FGF 7 and FGF 10 are involved in re-epithelialization by promoting the proliferation of keratinocytes, and also involved in maintaining keratinocytes by neutralizing reactive oxygen species.

Conventional technologies aimed at improving skin elasticity or wrinkles with collagen, and methods of promoting collagen synthesis are mainly characterized by strengthening the epidermal layer. However, there was a problem in that a skin elasticity or wrinkle improvement effect may not be exhibited when only the epidermal layer is strengthened in practical application to the skin. Therefore, there is an urgent need to develop a skin anti-aging composition that strengthens the dermal layer of skin and improves skin elasticity or wrinkles by strengthening the basement membrane of the skin.

### RELATED-ART DOCUMENTS

### Non-Patent Documents

1: Barrientos S, Stojadinovic O, Golinko M, Brem H, Vinay Tomic-Canic M. 2008. Growth factors and cytokines in wound healing. Wound Rep Reg. 16:585

### DISCLOSURE

### Technical Problem

As a result of efforts to find a composition that exhibits a skin elasticity or wrinkle improvement effect as an anti-aging effect by strengthening the basement membrane of the skin, the present inventors have found that cordycepin which is a compound represented by Chemical Formula 1, andrographolide which is a compound represented by Chemical Formula 2, and quercetin represented by Chemical Formula 3 exhibit a skin elasticity or wrinkle improvement effect, particularly, that when the compound represented by Chemical Formula 1; and the compound represented by Chemical Formula 2 and/or the compound represented by Chemical Formula 3 are used together, it is highly effective for strengthening the dermal layer of skin and improving skin elasticity and wrinkles, thereby completing the present invention.

Therefore, the present invention is directed to providing a cosmetic composition for improving skin elasticity or wrinkles, which includes, as an active ingredient: a compound represented by Chemical Formula 1 or a physiologically acceptable salt thereof; and a compound represented by Chemical Formula 2 or a physiologically acceptable salt thereof and/or a compound represented by Chemical Formula 3 or a physiologically acceptable salt thereof.

The present invention is also directed to providing a pharmaceutical composition for improving skin elasticity or wrinkles, which includes, as an active ingredient: a compound represented by Chemical Formula 1 or a pharmaceutically acceptable salt thereof; and a compound represented by Chemical Formula 2 or a pharmaceutically acceptable salt thereof and/or a compound represented by Chemical Formula 3 or a pharmaceutically acceptable salt thereof.

### Technical Solution

One aspect of the present invention provides a cosmetic composition for improving skin elasticity or wrinkles, which includes, as an active ingredient: a compound represented by Chemical Formula 1 or a physiologically acceptable salt thereof; and a compound represented by Chemical Formula 2 or a physiologically acceptable salt thereof and/or a compound represented by Chemical Formula 3 or a physiologically acceptable salt thereof.

Another aspect of the present invention provides a pharmaceutical composition for improving skin elasticity or wrinkles, which includes, as an active ingredient: a compound represented by Chemical Formula 1 or a pharmaceutically acceptable salt thereof; and a compound represented by Chemical Formula 2 or a pharmaceutically acceptable salt thereof and/or a compound represented by Chemical Formula 3 or a pharmaceutically acceptable salt thereof.

Still another aspect of the present invention provides a method of improving skin elasticity or wrinkles, which includes treating the skin with a cosmetic composition including, as an active ingredient: a compound represented by Chemical Formula 1 or a physiologically acceptable salt thereof; and a compound represented by Chemical Formula 2 or a physiologically acceptable salt thereof and/or a compound represented by Chemical Formula 3 or a physiologically acceptable salt thereof.

Yet another aspect of the present invention provides a method of improving skin elasticity or wrinkles, which includes administering a pharmaceutical composition including, as an active ingredient: a compound represented by Chemical Formula 1 or a pharmaceutically acceptable salt thereof; and a compound represented by Chemical Formula 2 or a pharmaceutically acceptable salt thereof and/or a compound represented by Chemical Formula 3 or a pharmaceutically acceptable salt thereof.

Yet another aspect of the present invention provides a use of a composition including, as an active ingredient: a compound represented by Chemical Formula 1 or a pharmaceutically acceptable salt thereof; and a compound represented by Chemical Formula 2 or a pharmaceutically acceptable salt thereof and/or a compound represented by Chemical Formula 3 or a pharmaceutically acceptable salt thereof for manufacturing a product for improving skin elasticity or wrinkles.

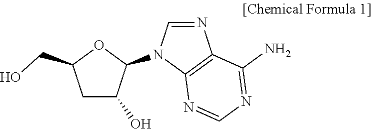 [SMILES: Nc1ncnc2c1ncn2[C@@H]1O[C@H](CO)C[C@H]1O]

The compound represented by Chemical Formula 1 has the molecular formula C₁₀H₁₃N₅O₃ and a molecular weight of 251.12 g/mol, and is named cordycepin, 3′-deoxyadenosine, 3′-deoxyadenosine 9-cordyceposidoadenine, 9-(3-deoxy-b-D-erythro-pentofuranosyl)-9Hpurin-6-amine, 9-(3-deoxy-b-D-ribofuranosyl)adenine, or the like.

In the present invention, a method of obtaining cordycepin is not particularly limited, and it may be chemically synthesized by a method known in the art, or a commercially available material may be used.

In the composition according to the present invention, the content of cordycepin may be 0.00001 to 10 wt %, for example, 0.00001 to 8 wt %, 0.00001 to 5 wt %, 0.00001 to 3 wt %, 0.00001 to 2 wt %, 0.00001 to 1 wt %, or 0.00002 to 1 wt %, relative to the total weight of the composition.

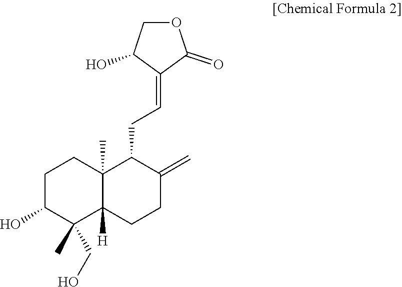 [SMILES: C=C1CC[C@@H]2[C@](C)(CO)[C@H](O)CC[C@@]2(C)[C@@H]1C/C=C1/C(=O)OC[C@H]1O]

The compound represented by Chemical Formula 2 has the molecular formula C₂₀H₃₀O₅ and a molecular weight of 350.45 g/mol, and is named andrographolide, 3-[2-[decahydro-6-hydroxy-5-(hydroxymethyl)-5,8a-dimethyl-2-methylene-1-naphthalenyl]ethylidene]dihydro-4-hydroxy-2(3H)-furanone, or the like.

In the present invention, a method of obtaining andrographolide is not particularly limited, and it may be chemically synthesized by a method known in the art, or a commercially available material may be used.

In the composition according to the present invention, the content of andrographolide may be 0.00001 to 10 wt %, for example, 0.00001 to 8 wt %, 0.00001 to 5 wt %, 0.00001 to 3 wt %, 0.00001 to 2 wt %, 0.00001 to 1 wt %, or 0.00002 to 1 wt %, relative to the total weight of the composition.

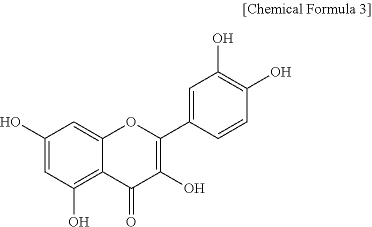 [SMILES: O=c1c(O)c(-c2ccc(O)c(O)c2)oc2cc(O)cc(O)c12]

The compound represented by Chemical Formula 3 has the molecular formula C₁₅H₁₀O₇ and a molecular weight of 302.236 g/mol, and is named quercetin, 2-(3,4-dihydroxyphenyl)-5,7-dihydroxy-4H-1-benzopyran-4-one, 3,3′,4′,5,7-pentahydroxyflavone, 5,7,3′,4′-flavon-3-ol, or the like.

In the present invention, a method of obtaining quercetin is not particularly limited, and it may be chemically synthesized by a method known in the art, or a commercially available material may be used.

In the composition according to the present invention, the content of quercetin may be 0.00001 to 10 wt %, for example, 0.00001 to 8 wt %, 0.00001 to 5 wt %, 0.00001 to 3 wt %, 0.00001 to 2 wt %, 0.00001 to 1 wt %, or 0.00002 to 1 wt %, relative to the total weight of the composition.

In addition, in the composition according to the present invention, the compound represented by Chemical Formula 1 or pharmaceutically acceptable salt thereof and the compound represented by Chemical Formula 2 or pharmaceutically acceptable salt thereof may be included in a weight ratio of 100:1 to 1:100 or 50:1 to 1:50. In the composition according to the present invention, the compound represented by Chemical Formula 1 or pharmaceutically acceptable salt thereof and the compound represented by Chemical Formula 3 or pharmaceutically acceptable salt thereof may be included in a weight ratio of 100:1 to 1:100 or 50:1 to 1:50. In the composition according to the present invention, the compound represented by Chemical Formula 1 or pharmaceutically acceptable salt thereof and the sum of the compound represented by Chemical Formula 2 or pharmaceutically acceptable salt thereof and the compound represented by Chemical Formula 3 or pharmaceutically acceptable salt thereof may be included in a weight ratio of 100:1 to 1:100 or 50:1 to 1:50.

In the present invention, a “skin elasticity improvement effect” refers to an action of preventing, suppressing, or inhibiting skin elasticity from being degraded or alleviating skin elasticity that has already degraded.

In the present invention, a “wrinkle improvement effect” refers to an action of preventing, suppressing, or inhibiting the formation of wrinkles on the skin or alleviating wrinkles that have already formed.

According to an embodiment of the present invention, it was confirmed that the mRNA expression of FGF 10 was promoted when human fibroblasts were treated with cordycepin, which is a compound represented by Chemical Formula 1, the mRNA expression of FGF 10 was promoted when human fibroblasts were treated with andrographolide, which is a compound represented by Chemical Formula 2, and the mRNA expression of FGF 10 was promoted when human fibroblasts were treated with quercetin, which is a compound represented by Chemical Formula 3. Also, it was confirmed that when human fibroblasts were treated with cordycepin in combination with andrographolide or quercetin, the mRNA expression level of FGF 10 was further increased compared to when cordycepin was used alone, andrographolide was used alone, and quercetin was used alone.

FGF 10 is expressed in fibroblasts that constitute the dermis and known to play a role in strengthening and normally maintaining the basement membrane of the skin. Also, FGF 10 serves to maintain skin integrity through signaling between the epidermis and dermis and is involved in the growth, division, adhesion, and migration of epidermal cells through interacting with heparin sulfate, which is a major proteoglycan constituting the basement membrane. Generally, the expression of FGF 10 is known to normally maintain the basement membrane itself and promote epidermal growth by transmitting signals through the basement membrane.

Therefore, the composition of the present invention promotes the proliferation of fibroblasts in the basement membrane of the skin through expression of FGF 10 to strengthen the skin basement membrane, and thus may exhibit a skin elasticity or wrinkle improvement effect.

Therefore, the composition of the present invention may include a compound represented by Chemical Formula 1 or a pharmaceutically acceptable salt thereof; and a compound represented by Chemical Formula 2 or a pharmaceutically acceptable salt thereof or a compound represented by Chemical Formula 3 or a pharmaceutically acceptable salt thereof, at the same time, and accordingly, exhibit a superior skin elasticity or wrinkle improvement effect compared to a composition including only a compound represented by Chemical Formula 1 or a pharmaceutically acceptable salt thereof, only a compound represented by Chemical Formula 2 or a pharmaceutically acceptable salt thereof, and only a compound represented by Chemical Formula 3 or a pharmaceutically acceptable salt thereof.

The composition according to the present invention may be prepared in the form of any one selected from the group consisting of a solution, an ointment for external use, a cream, a foam, a nutritional skin toner, a skin softening toner, a pack, softening water, a lotion, a makeup base, an essence, soap, a cleanser, bath salt, a sunscreen cream, a sunscreen oil, a suspension, an emulsion, a paste, a gel, a lotion, a powder, soap, a surfactant-containing cleansing, an oil, a powder foundation, an emulsion foundation, a wax foundation, a patch, and a spray, and preferably is in the form of a toner, an essence, a lotion, a cream, a pack, a gel, a powder, a foundation, or a cleanser, but the present invention is not limited thereto.

The cosmetic composition according to the present invention may further include one or more cosmetically acceptable carriers that are mixed in general skin cosmetics, and typical ingredients, for example, oil, water, a surfactant, a moisturizing agent, a lower alcohol, a thickening agent, a chelating agent, a pigment, a preservative, a fragrance, and the like, may be appropriately mixed, but the present invention is not limited thereto.

When the formulation of the present invention is a powder or spray, lactose, talc, silica, aluminum hydroxide, calcium silicate, polyamide powder, or a mixture thereof may be used as a carrier ingredient. Particularly, when the formulation is a spray, a propellant such as chlorofluorohydrocarbon, propane/butane, or dimethyl ether may be further included.

When the formulation of the present invention is a solution or emulsion, a solvent, a solubilizing agent, or an emulsifying agent may be used as a carrier ingredient. For example, water, ethanol, isopropanol, ethyl carbonate, ethyl acetate, benzyl benzoate, propylene glycol, or 1,3-butyl glycol oil may be used, and particularly, cottonseed oil, peanut oil, corn germ oil, olive oil, castor oil, sesame oil, aliphatic esters of glycerol, polyethylene glycol, or fatty acid esters of sorbitan may be used.

When the formulation of the present invention is a suspension, a liquid diluent such as water, ethanol, or propylene glycol, a suspending agent such as ethoxylated isostearyl alcohol, polyoxyethylene sorbitol ester, or polyoxyethylene sorbitan ester, microcrystalline cellulose, aluminum metahydroxide, bentonite, agar, tragacanth, or the like may be used as a carrier ingredient.

When the formulation of the present invention is a soap, an alkali metal salt of fatty acids, a fatty acid hemiester salt, a fatty acid protein hydrolysate, an isethionate, a lanolin derivative, an aliphatic alcohol, a vegetable oil, glycerol, sugar, or the like may be used as a carrier ingredient.

The present invention also provides a method of improving skin elasticity or wrinkles, which includes treating the skin of a subject in need with a cosmetic composition including, as an active ingredient, a compound represented by Chemical Formula 1 or a physiologically acceptable salt thereof; and a compound represented by Chemical Formula 2 or a physiologically acceptable salt thereof and/or a compound represented by Chemical Formula 3 or a physiologically acceptable salt thereof. The subject includes, without limitation, mammals including humans, livestock, rats, and the like.

The present invention also provides a use of a cosmetic composition including, as an active ingredient, a compound represented by Chemical Formula 1 or a pharmaceutically acceptable salt thereof; and a compound represented by Chemical Formula 2 or a pharmaceutically acceptable salt thereof and/or a compound represented by Chemical Formula 3 or a pharmaceutically acceptable salt thereof for manufacturing a product for improving skin elasticity or wrinkles.

Meanwhile, the pharmaceutical composition of the present invention may include an active ingredient alone and further include a pharmaceutically acceptable carrier, excipient, diluent, or accessory ingredient depending on the dosage form, method of use, and purpose of use.

More specifically, in addition to the active ingredient, a nutrient, a vitamin, an electrolyte, a flavoring agent, a coloring agent, a filler, pectic acid and a salt thereof, alginic acid and a salt thereof, an organic acid, a protective colloidal thickening agent, a pH controlling agent, a stabilizer, a preservative, glycerin, an alcohol, a carbonizing agent used in carbonated beverages, or the like may be further included.

The “pharmaceutically acceptable” means that it is physiologically acceptable and does not typically cause gastrointestinal disorders, allergic reactions such as dizziness, or similar reactions when administered to animals, preferably, humans. The “pharmaceutically effective amount” may appropriately vary depending on the type and severity of a disease to be treated, the age, weight, health condition, and sex of a patient, an administration route, a treatment time, and the like.

The pharmaceutically acceptable carrier, excipient, or diluent may be, for example, one or more selected from the group consisting of lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, mineral oil, dextrin, calcium carbonate, propylene glycol, liquid paraffin, and saline, but the present invention is not limited thereto. All typical carriers, excipients, or diluents may be used.

The above ingredients may be added independently or in combination with the active ingredient of the present invention.

The formulation of the pharmaceutical composition may vary depending on the method of use, and the pharmaceutical composition may be formulated using a method well known in the art to which the present invention pertains so that rapid, sustained, or delayed release of the active ingredient is provided after administration to a mammal. The formulation may be, for example, selected from the group consisting of an ointment, a cream, a tablet, a pill, a powder, a granule, a capsule, a suspension, a liquid for internal use, an emulsion, a syrup, an aqueous solution, a non-aqueous solvate, a suspension, an emulsion, and a patch.

For the formulation, a typical excipient, for example, a filler, an extending agent, a binding agent, a disintegrating agent, a surfactant, an anti-agglomerating agent, a lubricant, a wetting agent, a flavoring agent, an emulsifying agent, a preservative, a sweetening agent, a fragrance, a preservative, or the like may be further included.

In general, a solid preparation for oral administration includes a tablet, a pill, a soft or hard capsule, a pill, a powder, a granule, and the like, and may be prepared by mixing with one or more excipients, for example, starch, calcium carbonate, sucrose or lactose, gelatin, and the like. In addition to the simple excipient, a lubricant such as magnesium stearate or talc may also be used.

In addition, a liquid preparation for oral administration includes a suspension, a liquid for internal use, an emulsion, a syrup, and the like. In addition to the generally used simple diluent such as water and liquid paraffin, various excipients such as a wetting agent, a sweetening agent, a fragrance, a preservative, and the like may be included.

Examples that may be mentioned for dermal administration include formulations suitable for producing a dusting powder, an emulsion, a suspension, an oil, a spray, an ointment, a grease ointment, a cream paste, a gel, a foam, or a solution, and carriers and/or excipients suitable for transdermal therapeutic systems (TTSs) may be included. The composition of the present invention may be in the form of a semi-solid, particularly, an ointment (solution ointment, suspension ointment), a cream, a gel, or a paste. Examples of the ingredient commonly used in the oil phase include a fatty alcohol such as lauryl alcohol, cetyl alcohol, or stearyl alcohol, a fatty acid such as palmitic acid or stearic acid, liquid or solid paraffin, ozokerite, liquid or solid wax such as isopropyl myristate, and a natural fat or partially synthetic fat such as coconut fatty acid triglyceride, a hydrogenated oil such as hydrogenated peanut or castor oil, or a partial fatty ester of glycerol such as glycerol monostearate or glycerol distearate. Examples of the emulsifying agent include a surfactant such as a non-ionic surfactant such as a polyalcohol or a fatty acid ester of an ethylene oxide adduct thereof such as polyglycerol fatty acid ester or polyoxyethylene sorbitan fatty acid ester, a sorbitan fatty acid ester such as sorbitan oleate or sorbitan isostearate, isostearate, a sterol, a polyoxyethylene fatty alcohol ether, or a fatty acid ester, and an anionic surfactant such as an alkali metal salt of fatty alcohol sulfonates such as sodium lauryl sulfate, sodium cetyl sulfate, and sodium stearyl sulfate, and they may be commonly used in the presence of the fatty alcohol such as cetyl alcohol or stearyl alcohol. Among those listed above, particularly, a preparation for preventing cream drying, for example, a polyalcohol such as glycerol, sorbitol, propylene glycol, or polyethylene glycol, may be added to an aqueous phase, or a preservative, a fragrance, or the like may be added to an aqueous phase.

The pharmaceutical composition of the present invention may be in the form of an anhydrous ointment and may contain paraffin, particularly, low viscous paraffin, which is suitable for local application and is liquid at body temperature, or contain the above-mentioned natural fat or partially synthetic fat such as coconut fatty acid triglyceride, a hydrogenated oil such as hydrogenated peanut or castor oil, or a partial fatty ester of glycerol such as glycerol monostearate or glycerol distearate, a silicone such as polymethylsiloxane such as hexamethyldisiloxane or octamethyltrisiloxane. Also, for example, a fatty alcohol, a sterol, wool wax, other emulsifying agents, and/or other additives, which are associated with a water-based cream and increase water absorption capacity, may be contained.

In the present invention, when the pharmaceutical composition is formulated into a drug, reference may be made to the contents disclosed in the Remington's document (Remington's Pharmaceutical Science, Mack Publishing Company, Easton PA), and the above document is included as a part of the present specification.

The present invention also provides a method of improving skin elasticity or wrinkles, which includes administering, to a subject in need, a pharmaceutical composition including, as an active ingredient: a compound represented by Chemical Formula 1 or a pharmaceutically acceptable salt thereof; and a compound represented by Chemical Formula 2 or a pharmaceutically acceptable salt thereof and/or a compound represented by Chemical Formula 3 or a pharmaceutically acceptable salt thereof. The subject includes, without limitation, mammals including humans, livestock, rats, and the like.

The present invention also provides a use of a pharmaceutical composition including, as an active ingredient: a compound represented by Chemical Formula 1 or a pharmaceutically acceptable salt thereof; and a compound represented by Chemical Formula 2 or a pharmaceutically acceptable salt thereof and/or a compound represented by Chemical Formula 3 or a pharmaceutically acceptable salt thereof for manufacturing a product for improving skin elasticity or wrinkles.

In the present invention, the term “pharmaceutical composition” may be used as a concept including the meaning of a “quasi-drug” or “drug.”

When the composition of the present invention is used as an external preparation for skin, an adjuvant typically used in the field of dermatology, such as a lipid material, an organic solvent, a solubilizing agent, a concentrating agent, a gelating agent, an emollient, an antioxidant, a suspending agent, a stabilizer, a foaming agent, a fragrance, a surfactant, water, an ionic or non-ionic emulsifying agent, a filler, a sequestering agent, a chelating agent, a preservative, a vitamin, a blocking agent, a wetting agent, an essential oil, a dye, a pigment, a hydrophilic or lipophilic active agent, a lipid vesicle, and any other ingredients typically used in an external preparation for skin may be further included. Also, the above ingredients may be added in amounts typically used in the field of dermatology.

In addition, when the composition of the present invention is provided as a composition for external use, it may be in the form of an ointment, a patch, a gel, a cream, or a spray, but the present invention is not limited thereto.

The composition for external use of the present invention may be used as a parenteral preparation. For example, the composition for external use may be prepared by a typical method of preparing a composition for external skin application, in which a pharmaceutically acceptable base such as Vaseline, stearyl alcohol, or the like; a pharmaceutically acceptable surfactant such as polysorbate, sorbitan sesquioleate, or the like; a pharmaceutically acceptable moisturizing agent such as glycerin or the like; a pharmaceutically acceptable solvent; and a fragrance, a coloring agent, a stabilizer, a thickening agent, and the like are homogeneously mixed.

When the composition of the present invention is provided as a quasi-drug composition, one or more compounds selected from the group consisting of hydroxycinnamic acid, isoamyl acetate, and betaine or a pharmaceutically acceptable salt thereof may be included as an active ingredient. In addition, as needed, a pharmaceutically acceptable carrier, excipient, or diluent may be further included. The pharmaceutically acceptable carrier, excipient, or diluent is not limited as long as it does not impair the effects of the present invention, and for example, a filler, an extending agent, a binding agent, a wetting agent, a disintegrating agent, a surfactant, a lubricant, a sweetening agent, a fragrance, a preservative, and the like may be included.

As the quasi-drug composition, a disinfectant cleanser, a shower foam, an ointment, a wet tissue, a coating agent, and the like may be exemplified. Preferably, the quasi-drug composition may be prepared in a semi-solid preparation such as an ointment for external use or a lotion, but the present invention is not limited thereto. The preparation method, capacity, method of use, and ingredients of the quasi-drug may be appropriately selected from typical techniques known in the art.

In the present invention, the term “quasi-drug” refers to a product that has a milder effect on the human body than a drug despite exhibiting the effect of treating, alleviating, or preventing a disease. The quasi-drug excludes products used for pharmaceutical purposes under the Pharmaceutical Affairs Act and includes products according to the classification standards separately prescribed by the Ministry of Health and Welfare. Specifically, the quasi-drug may be an external preparation for skin or a personal hygiene product, but the present invention is not limited thereto.

The above-described ingredients included in the cosmetic composition according to the present invention may be included in the cosmetic composition of the present invention within a range not exceeding the maximum amount specified in standards related to “Cosmetic Use and Permission” prescribed by each government. For example, it may comply with the scope specified in the “Cosmetics Safety Technical Specification” prescribed by China.

Numerical values described herein above should be construed as including equivalent ranges unless otherwise specified.

### Advantageous Effects

The present invention provides a composition including a compound represented by Chemical Formula 1; and a compound represented by Chemical Formula 2 and/or a compound represented by Chemical Formula 3, at the same time. The composition has a superior skin elasticity or wrinkle improvement effect compared to a composition including only a compound represented by Chemical Formula 1, only a compound represented by Chemical Formula 2, or only a compound represented by Chemical Formula 3.

### DESCRIPTION OF DRAWINGS

FIG. 1 is a set of images for comparing cases in which the basement membrane is subjected to UV irradiation and then treated/not treated with cordycepin and andrographolide.

### MODES OF THE INVENTION

Hereinafter, the present invention will be described in detail with reference to the following examples. However, it should be understood that the following examples are given for the purpose of illustration only and are not intended to limit the scope of the present invention.

### Experimental Example 1. Analysis of mRNA Expression of FGF10

Human dermal fibroblasts (neonatal) were purchased from Lonza and then cultured in a carbon dioxide incubator at 37° C. and 5% carbon dioxide. In this case, as a medium for culturing, a medium containing Dulbecco's modified eagle medium (DMEM; Thermo Fisher Scientific) with 10% fetal bovine serum (FBS) and 1% antibiotics (penicillin/streptomycin) was used. Afterward, 20000 cells were seeded in a 6-well plate, and 24 hours later, the DMEM culture medium was treated with each sample having compositions shown in Tables 1 and 2 and cultured again for another 48 hours. As a control, a DMEM culture medium not including any other components was used. Afterward, intracellular RNA was purified, cDNA was synthesized, and the mRNA expression level of FGF10 was analyzed by a qPCR method. Table 1 shows the expression levels when cordycepin and andrographolide are used alone or in combination compared to the control. Table 2 shows the expression levels when cordycepin and quercetin are used alone and or combination compared to the control. Adenosine is a substance well known to have a skin wrinkle improvement effect in the related art, but it was confirmed to have no effect on increasing the expression of FGF 10.

TABLE 1

| | Expression level |
|---|---|
| Sample and concentration | (% relative to control) |
| Cordycepin 8 ppm | 148 |
| Andrographolide 0.2 ppm | 141 |
| Cordycepin 8 ppm + Andrographolide 0.2 ppm | 536 |
| Adenosine 8 ppm | 88 |

TABLE 2

| | [symbol] Expression level |
|---|---|
| Sample and concentration | (% relative to control) |
| Cordycepin 0.2 ppm | 102 |
| Quercetin 0.2 ppm | 63 |
| Cordycepin 0.2 ppm + Quercetin 0.2 ppm | 234 |
| Adenosine 8 ppm | 88 |
| Adenosine 8 ppm + Quercetin 0.2 ppm | 59 |

### Experimental Example 2. Analysis of Basement Membrane Change Using Pig Skin

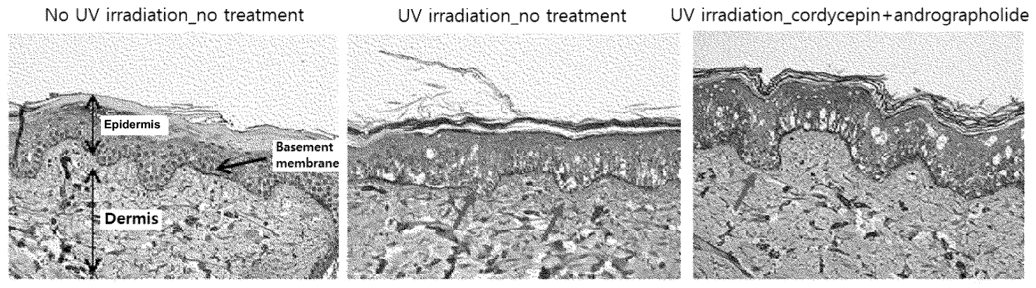

Live pig skin (used in the experiment immediately after slaughter, without freezing) was purchased from Apures and then cultured in a carbon dioxide incubator at 37° ° C. and 5% carbon dioxide using a medium containing DMEM (Thermo Fisher Scientific) with 10% FBS, 1% antibiotics (penicillin/streptomycin), and 0.02% tetracycline. The pig skin was stabilized in a 6-well hanging insert (MILLIPORE) for 24 hours and then subjected to UVB irradiation at an intensity of 1 J/cm² using a UV irradiation device to induce breakdown of the basement membrane. Here, UV irradiation was performed a total of three times with 5-minute irradiation followed by a 2-hour break. Immediately after the UVB irradiation, a sample including 0.1% cordycepin and 0.1% andrographolide was applied onto the pig skin surface, and the pig skin was treated for 48 hours. Afterward, the sample was fixed with 10% formalin, and TEGO SCIENCE was commissioned to conduct H&E staining. During the experimental period, DMEM was exchanged once in the morning and once in the afternoon. Experimental results are shown in FIG. 1. It can be confirmed that, when the basement membrane was subjected to UV irradiation, the basement membrane was flattened, and when cordycepin and andrographolide were applied thereto, the flattened basement membrane was restored to its original state.

### Experimental Example 3. Evaluation of Elasticity or Wrinkle Improvement Effect on Human Skin

In order to evaluate a skin improvement effect resulting from a basement membrane strengthening effect on human skin, a preparation example was prepared as shown in the following table (units: wt %).

TABLE 3
Preparation Example

| | |
|---|---|
| Cordycepin | 0.002 |
| Quercetin | 0.002 |
| Water | remainder |
| Glycerin | 8.0 |
| Butylene glycol | 4.0 |
| Hyaluronic acid extract | 5.0 |
| β-Glucan | 7.0 |
| Carbomer | 0.15 |
| Caprylic/capric triglyceride | 8.0 |
| Squalane | 5.0 |
| Cetearyl glucoside | 1.5 |
| Sorbitan stearate | 0.4 |
| Cetearyl alcohol | 2.0 |
| Preservative | adequate amount |
| Pigment | adequate amount |
| Triethanolamine | 0.15 |

32 women aged 27 to 43 were asked to use the cosmetic according to the preparation example of Table 3 on their facial area once every morning and every evening, that is, twice a day, for a total of 4 weeks, and the conditions of skin wrinkles and skin elasticity were measured. Measurement was made once immediately before the first day of use (A) and once after 4 weeks of use (B), and the measurement method is as follows. For skin wrinkle measurement, residual wrinkles (potential wrinkles) formed by facial expression were measured once in the test area (around the eyes) using an Antera 3D CS instrument (Miravex, Ireland), and the depth (mm) of the Wrinkle-small analysis mode was used as evaluation data. The test subject was asked to create intentional wrinkles around the eyes by frowning and maintain the facial wrinkles for 1 minute, and immediately after relaxing her facial expression, the remaining wrinkles were measured. The measurement device is a device that is able to measure a skin surface image using a light-emitting diode (LED light source), and data was extracted from the 3D shape image of the built-in program, skin conditions were quantified, and the image was utilized. A decrease in the wrinkle depth value (mm) after 4 weeks of use compared to that before use of the product means that residual wrinkles (potential wrinkles) formed by facial expression are improved. Skin elasticity was measured using a dermal torque meter (Dia-Stron, United Kingdom), the test area (cheek) was measured three times, and the average value was used as evaluation data. Measurement was made by a torsion method between a 3 mm guard ring and a central disk. Ur/Ue values were used as indicators for evaluating internal skin elasticity, and an increase in measurement values means that internal skin elasticity is improved.

The measurement results were substituted into the following equation to calculate improvement rates, and the improvement rates are shown in the following Table 4.

$$Improvement rate = \{ \frac{\begin{matrix}Measurement value after use ( B ) - \\ Measurement value before use ( A )\end{matrix}}{Measurement value before use ( A )} \} \times 100$$

TABLE 4

| Evaluation items | Improvement rate (%) |
|---|---|
| Wrinkle improvement | 10.5 |
| Elasticity improvement | 12.2 |

1. A cosmetic composition for improving skin elasticity or wrinkles, comprising, as an active ingredient:
a compound represented by the following Chemical Formula 1 or a physiologically acceptable salt thereof; and
a compound represented by the following Chemical Formula 2 or a physiologically acceptable salt thereof and/or a compound represented by the following Chemical Formula 3 or a physiologically acceptable salt thereof:

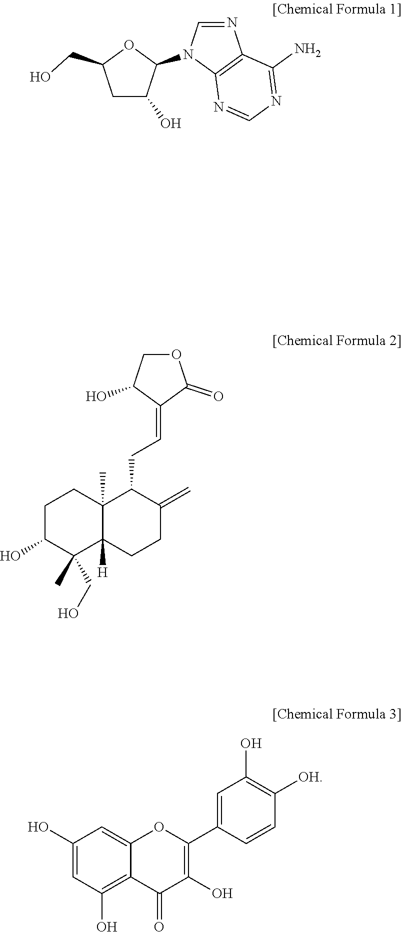 [SMILES: Nc1ncnc2c1ncn2[C@@H]1O[C@H](CO)C[C@H]1O ; C=C1CC[C@@H]2[C@](C)(CO)[C@H](O)CC[C@@]2(C)[C@@H]1C/C=C1/C(=O)OC[C@H]1O ; O=c1c(O)c(-c2ccc(O)c(O)c2)oc2cc(O)cc(O)c12]

2. The cosmetic composition of claim 1, wherein the compound represented by Chemical Formula 1 or physiologically acceptable salt thereof is included in an amount of 0.00001 wt % to 10 wt % of the entire composition, and
the compound represented by Chemical Formula 2 or physiologically acceptable salt thereof and/or the compound represented by Chemical Formula 3 or physiologically acceptable salt thereof is/are included in an amount of 0.00001 wt % to 10 wt % of the entire composition.

3. The cosmetic composition of claim 1, wherein the compound represented by Chemical Formula 1 or physiologically acceptable salt thereof; and the compound represented by Chemical Formula 2 or physiologically acceptable salt thereof and/or the compound represented by Chemical Formula 3 or physiologically acceptable salt thereof are included in a weight ratio of 100:1 to 1:100.

4. The cosmetic composition of claim 1, wherein the composition has an effect of strengthening the basement membrane of the skin.

5. A pharmaceutical composition for improving skin elasticity or wrinkles, comprising, as an active ingredient:
a compound represented by the following Chemical Formula 1 or a pharmaceutically acceptable salt thereof; and
a compound represented by the following Chemical Formula 2 or a pharmaceutically acceptable salt thereof and/or a compound represented by the following Chemical Formula 3 or a pharmaceutically acceptable salt thereof:

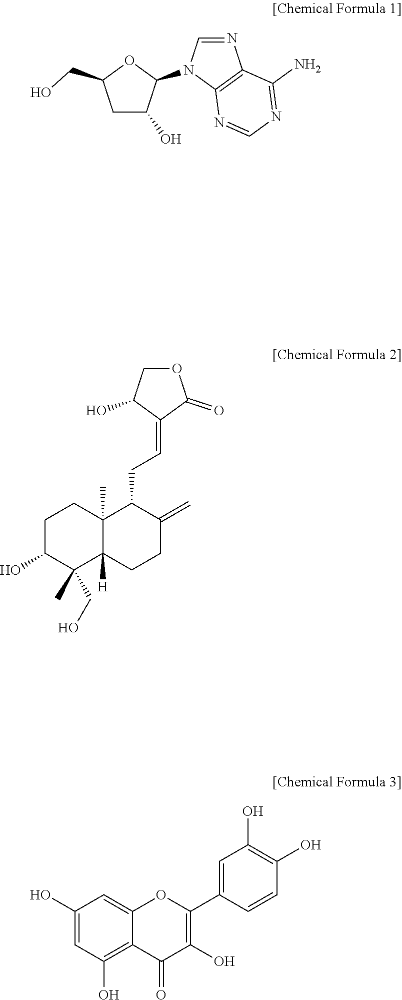 [SMILES: Nc1ncnc2c1ncn2[C@@H]1O[C@H](CO)C[C@H]1O ; C=C1CC[C@@H]2[C@](C)(CO)[C@H](O)CC[C@@]2(C)[C@@H]1C/C=C1/C(=O)OC[C@H]1O ; O=c1c(O)c(-c2ccc(O)c(O)c2)oc2cc(O)cc(O)c12]

6. The pharmaceutical composition of claim 5, wherein the compound represented by Chemical Formula 1 or pharmaceutically acceptable salt thereof is included in an amount of 0.00001 wt % to 10 wt % of the entire composition, and
the compound represented by Chemical Formula 2 or pharmaceutically acceptable salt thereof and/or the compound represented by Chemical Formula 3 or pharmaceutically acceptable salt thereof is/are included in an amount of 0.00001 wt % to 10 wt % of the entire composition.

7. The pharmaceutical composition of claim 5, wherein the compound represented by Chemical Formula 1 or pharmaceutically acceptable salt thereof; and the compound represented by Chemical Formula 2 or pharmaceutically acceptable salt thereof and/or the compound represented by Chemical Formula 3 or pharmaceutically acceptable salt thereof are included in a weight ratio of 100:1 to 1:100.

8. The pharmaceutical composition of claim 5, wherein the composition has an effect of strengthening the basement membrane of the skin.

9. A method of improving skin elasticity or wrinkles comprising applying to the skin of a subject in need thereof a composition comprising, as an active ingredient:
a compound represented by Chemical Formula 1 or a physiologically acceptable salt thereof; and a compound represented by Chemical Formula 2 or a physiologically acceptable salt thereof and/or a compound represented by Chemical Formula 3 or a physiologically acceptable salt thereof: 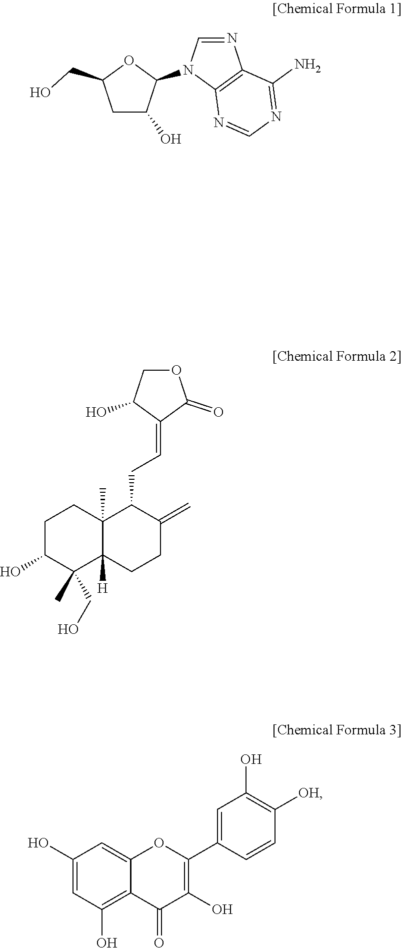 [SMILES: Nc1ncnc2c1ncn2[C@@H]1O[C@H](CO)C[C@H]1O ; C=C1CC[C@@H]2[C@](C)(CO)[C@H](O)CC[C@@]2(C)[C@@H]1C/C=C1/C(=O)OC[C@H]1O ; O=c1c(O)c(-c2ccc(O)c(O)c2)oc2cc(O)cc(O)c12]
and wherein the composition is a cosmetic composition or pharmaceutical composition.

10. The method of claim 9, wherein the compound represented by Chemical Formula 1 or physiologically acceptable salt thereof is included in an amount of 0.00001 wt % to 10 wt % of the entire composition, and
the compound represented by Chemical Formula 2 or physiologically acceptable salt thereof and/or the compound represented by Chemical Formula 3 or physiologically acceptable salt thereof is/are included in an amount of 0.00001 wt % to 10 wt % of the entire composition.

11. The method of claim 9, wherein the compound represented by Chemical Formula 1 or physiologically acceptable salt thereof; and the compound represented by Chemical Formula 2 or physiologically acceptable salt thereof and/or the compound represented by Chemical Formula 3 or physiologically acceptable salt thereof are included in a weight ratio of 100:1 to 1:100.

12. The method of claim 9, wherein the composition has an effect of strengthening the basement membrane of the skin.
